(19) United States
(12) Patent Application Publication
(10) Pub. No.: US 2008/0070982 A1
(43) Pub. Date: Mar. 20, 2008

(54) System and methods of melanoma prevention

(76) Inventors: Graham Timmins, Albuquerque, NM (US); Leslie Paige Lund, Albuquerque, NM (US)

Correspondence Address: VALAUSKAS & PINE LLC, Suite 1825, 150 North Wacker Drive, Chicago, IL, 60606, US

(21) Appl. No.: 11/901,056

(22) Filed: Sep. 14, 2007

Related U.S. Application Data
(60) Provisional application No. 60/844,754, filed on Sep. 15, 2006.

(51) Int. Cl. A61K 31/351; A61P 17/16; A61Q 17/04

(52) U.S. Cl. 514/460

(57) ABSTRACT

Inhibiting the enzymic action of tyrosinase in the melanocyte prior to, during, or after ultraviolet (UV) radiation exposure, including over-exposure causing erythema, or sunburn, prevents the production of melanin and thereby melanoma. Melanoma can be prevented by using a tyrosinase inhibitor agent that inhibits the enzymic action of tyrosinase to prevent damage and/or death of melanocytes. The inhibitor agent can be applied to the skin or ingested.

This application claims the benefit of U.S. Provisional Application No. 60/844,754 filed Sep. 15, 2006.

### FIELD OF THE INVENTION

The present invention relates generally to tyrosinase inhibition, and more particularly to preventing melanoma due to ultraviolet (UV) radiation exposure.

### BACKGROUND OF THE INVENTION

Melanocytes are typically located in the bottom layer of the epidermis, or outermost layer of the skin. A melanocyte, also referred to herein as cell, produces pigment. The pigment, or color, that melanocytes make is called melanin. The major determinant of color is not the number, but rather the tyrosinase activity within the melanocytes. Melanin production takes place in unique organelles (tiny structures within the cell) known as melanosomes. Darkly pigmented skin, hair and eyes have melanosomes that contain more melanin.

Melanosomes rearrange themselves within the cell in response to external cues such as ultraviolet (UV) radiation. Melanosomes usually cluster together near the center of the cell but can rapidly redistribute themselves. Melanosome redistribution involves supplying melanin from the melanocyte to nearby keratinocytes. A keratinocyte is the major cell type of the epidermis, making up about 90% of epidermal layer.

Tyrosinase is an enzyme found in a melanosome of a melanocyte. Tyrosinase catalyzes the production of melanin and other pigments from the amino acid tyrosine by oxidation. On a specific level, tyrosinase first transforms tyrosine into 3,4-dihydroxyphenylalanine (DOPA), then to the corresponding quinone (DOPAquinone), and finally to 2-carboxy-2,3-dihydroindole-5,6-quinone (DOPAchrome), which is further converted by other enzymes to yet more highly oxidized materials including melanin. Thus, skin pigmentation, or melanin production, depends upon the activity of tyrosinase. If this enzyme is not active, pigmentation does not occur.

The sun emits ultraviolet (UV) radiation in the form of A rays (UVA), B rays (UVB), and C rays (UVC). The approximate wavelength band, or range, in nanometers for UVA is 400-320 nm, UVB is 320-280 nm, and UVC is below 280 nm. Erythema, or sunburn, is an abnormal redness of the skin caused by over-exposure to ultraviolet (UV) radiation. This condition occurs when incident ultraviolet (UV) radiation exceeds the protective capacity of melanin in the skin. Sunburn can be life-threatening and is a leading cause of skin cancer, such as melanoma. Melanoma is a malignant tumor that arises from a mutant melanocyte.

Although melanoma is one of the rarer types of skin cancer, it causes the majority of skin cancer related deaths. Melanoma rates have been increasing over the years. Increases in melanoma were 2.5% annually between 1992 and 2001. Melanoma has a disproportionately high mortality in younger age groups, such as 18 to 40 year olds, with each death resulting in a loss of almost 19 years of expected life, among the highest for adult onset cancers.

When exposed to ultraviolet (UV) radiation, tryosinase activity results causing deoxyribonucleic acid (DNA) damage to melanocytes, thereby increasing the risk of melanoma.

The correlation between tyrosinase inhibition and protection of skin against unwanted pigmentation is accepted by the medical profession and by the cosmetic industry. Tyrosinase inhibitor agents, for example, vitamin C, gluthathione, kojic acid, placenta extract, sulfite salts and cysteine, have been used in order to inhibit tyrosinase activity to prevent melanin production such as to produce a pigmentation-lightened skin, but these agents have not been used to prevent melanoma resultant from ultraviolet (UV) radiation exposure.

There is a demand for a system and methods to prevent melanoma due to ultraviolet (UV) radiation exposure. The present invention satisfies this demand.

### SUMMARY OF THE INVENTION

The present invention prevents melanoma resultant from ultraviolet (UV) radiation exposure. The interaction of ultraviolet (UV) radiation with melanin in the melanosome generates an action spectrum that is identical to that of melanoma. Skin melanoma is a site of localized activity of tyrosinase, and is often associated with cancerous cell modification. In other words, the release of the enzyme tyrosinase in the melanosomes of the melanocytes causes damage and/or death of the melanocyte.

Inhibiting the enzymic action of tyrosinase in the melanocyte prior to ultraviolet (UV) radiation exposure, during ultraviolet (UV) radiation exposure, or after ultraviolet (UV) radiation exposure, including over-exposure causing erythema, or sunburn, prevents melanoma that would otherwise result. Melanoma can be prevented by using a tyrosinase inhibitor agent to prevent damage and/or death of melanocytes. The tyrosinase inhibitor agent can be applied to the skin or ingested.

An object of the present invention is to prevent melanoma.

Another object of the present invention is to inhibit the enzymic action of tyrosinase.

Yet another object of the present invention is to prevent damage and/or death of melanocytes.

Yet another object of the present invention is to prevent damage and/or death of melanocytes prior to exposure to ultraviolet (UV) radiation by using a tyrosinase inhibitor agent.

Yet another object of the present invention is to prevent damage and/or death of melanocytes during exposure to ultraviolet (UV) radiation by using a tyrosinase inhibitor agent.

Yet another object of the present invention is to prevent damage and/or death of melanocytes after exposure to ultraviolet (UV) radiation by using a tyrosinase inhibitor agent.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

FIG. 1 is a schematic of tyrosinase mediated DNA damage;

FIG. 2 is a schematic of a tyrosinase inhibitor preventing DNA damage;

FIG. 3 is a graph illustrating death of melanocytes with tyrosinase inhibitors applied after ultraviolet (UV) radiation exposure; and

FIG. 4 is a graph illustrating a DNA adduct assay.

### DETAILED DESCRIPTION OF EMBODIMENTS OF THE INVENTION

A melanocyte, or cell, produces melanin within tiny structures known as melanosomes. Tyrosinase is an enzyme found in a melanosome of a melanocyte. Tyrosinase activity includes catalyzing the production of melanin and other pigments from the amino acid tyrosine by oxidation. Thus, the production of melanin depends upon the activity of tyrosinase. If this enzyme is not active, pigmentation does not occur.

The present invention prevents melanoma prior to, during, or after ultraviolet (UV) radiation exposure. The interaction of ultraviolet (UV) radiation with melanin in the melanosome generates an action spectrum that is identical to that of melanoma. When melanocytes are exposed to ultraviolet (UV) radiation, tyrosinase activity within the melanosome occurs by oxidation that further produces reactive species that cause damage, and possibly death to the melanocytes, thereby increasing the risk of melanoma.

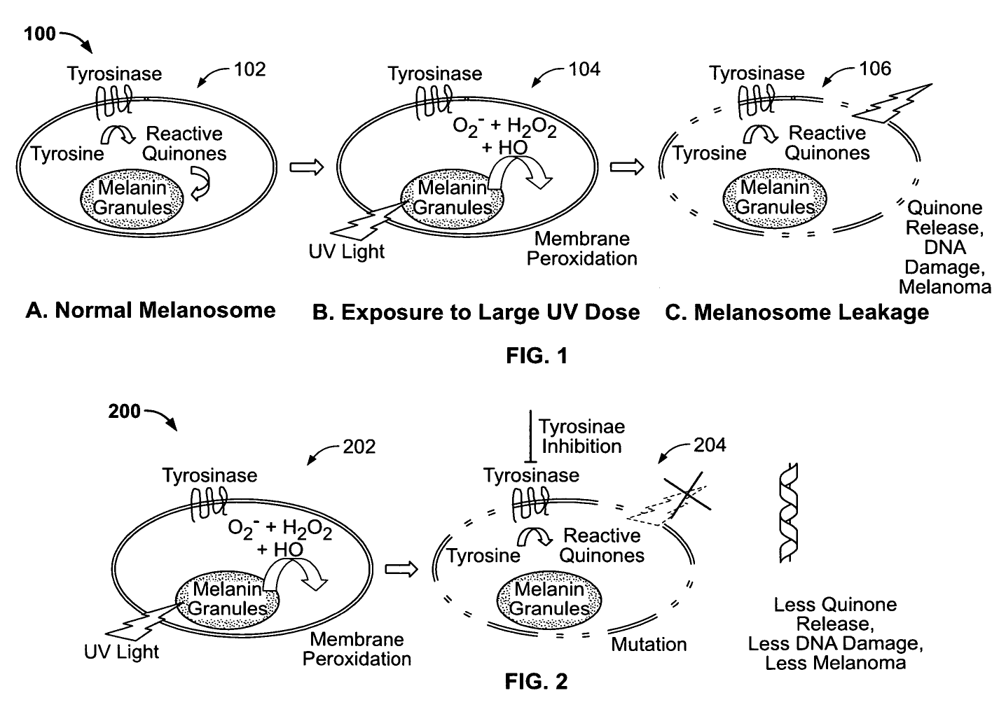

FIG. 1 is a schematic of tyrosinase mediated DNA damage. Melanosome 102 is considered normal and includes tyrosinase-derived reactive quinones that form melanin. Melanosome 104 is exposed to a high flux ultraviolet (UV) radiation, which activates melanin as a photosensitizer. The ultraviolet (UV) radiation produces oxidants leading to melanosome membrane disruption, or membrane peroxidation. As a result, reactive quinones pass out of the melanosome 106, damaging DNA.

Skin melanoma is a site of localized activity by tyrosinase, and is often associated with cancerous cell modification. In summary, the release of the enzyme tyrosinase in the melanosomes of the melanocytes causes damage and/or death of the melanocyte.

Inhibiting the enzymic action of tyrosinase in the melanocyte prior to, during, or after ultraviolet (UV) radiation exposure, including over-exposure causing erythema, or sunburn, prevents the production of melanin and thereby melanoma. Melanoma can be prevented by using a tyrosinase inhibitor agent to prevent damage and/or death of melanocytes. The inhibitor agent can be applied to the skin or ingested.

FIG. 2 illustrates how tyrosinase inhibition assists in preventing DNA damage and ultimately melanoma. A tyrosinase inhibitor is applied prior to, during, or after ultraviolet (UV) exposure. Melanosome 202 is exposed to a high flux ultraviolet (UV) radiation, which activates melanin as a photosensitizer. The ultraviolet (UV) radiation exposure produces oxidants leading to melanosome membrane disruption, or membrane peroxidation. Melanosome 204 includes a tyrosinase inhibitor agent such that less quinones pass out of the melanosome 204, resulting is less DNA damage and ultimately preventing melanoma.

Topically applied tyrosinase inhibitor agents are already widely used for skin lightening purposes. Treatments currently exist for ultraviolet (UV) radiation exposure such as sunburn gels, lotions and creams that are applied topically post-exposure. The present invention contemplates combining ultraviolet (UV) radiation post-exposure treatments with tyrosinase inhibitor agents. Tyrosinase inhibitor agents that can be topically applied to the skin include, for example gentisic acid and its esters, kojic acid and its esters, hydroquinone, and deoxyarbutin. The present invention also contemplates ingesting a tyrosinase inhibitor agent.

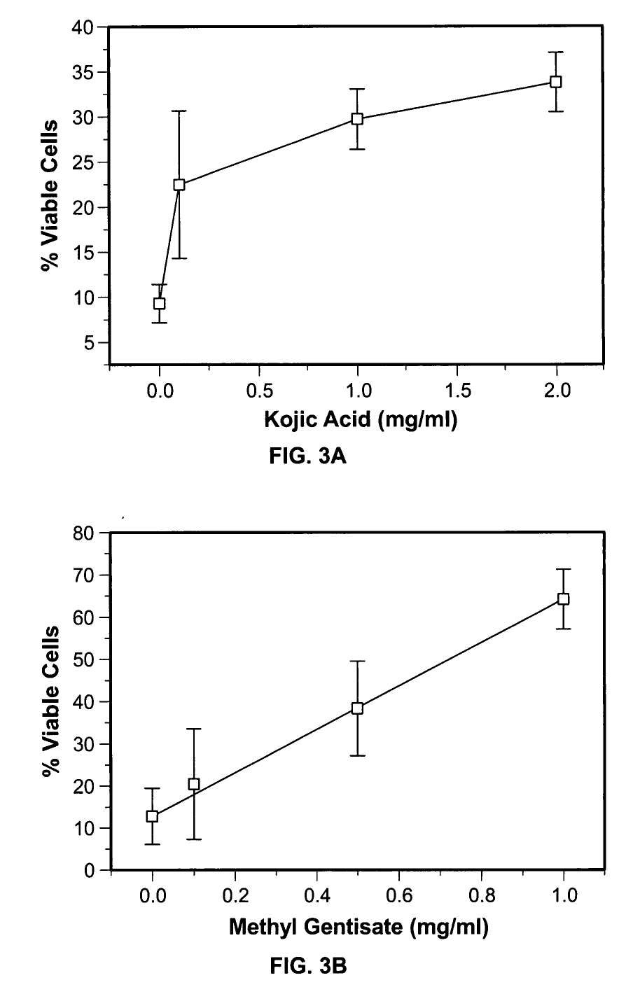

FIG. 3 is a graph illustrating death of melanocytes with tyrosinase inhibitors applied subsequent to ultraviolet (UV) radiation exposure. The graph illustrates results from a study conducted with mice melanocyte cell line (B16). The mouse melanocyte cell line (B16) was exposed to solar simulated ultraviolet (UV) radiation for 20 minutes. The solar ultraviolet (UV) radiation was simulated using a 300 W Xe solar simulator, equivalent to approximately 15 Minimal Erythemal Doses (MEDs), or equivalent to strong sunburn. After ultraviolet (UV) radiation exposure, cells were treated with increasing doses of the following tyrosinase inhibitors: kojic acid (shown in FIG. 3A) and methyl gentisate (shown in FIG. 3B). Melanocyte survival was determined by trypan blue exclusion 24 hours after ultraviolet (UV) radiation exposure. It was shown that there was no effect of tyrosinase inhibitors on the survival in non-ultraviolet (UV) exposed cells (not shown in FIG. 3). However, a dose dependent increase in viability was seen in treatment with a tyrosinase inhibitor in ultraviolet (UV) exposed cells. Preventing quinones from passing out of the melanosome substantially protects melanocytes from DNA damage.

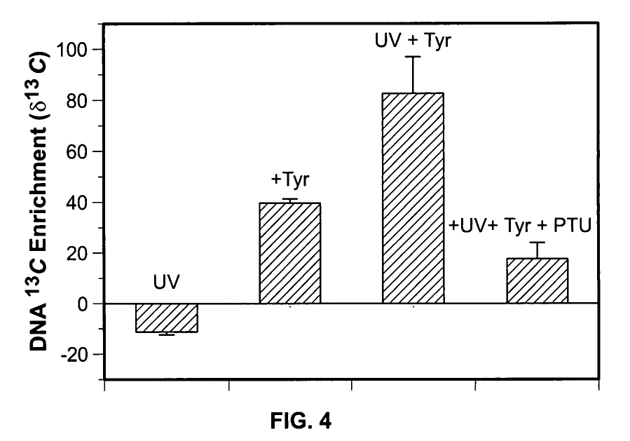

FIG. 4 is a graph illustrating a DNA adduct assay. Mouse melanocyte cell line (B16) was incubated with 300 μM phenylthiourea (PTU) immediately after ultraviolet (UV) radiation exposure. After 20 minutes of full spectrum ultraviolet (UV) radiation exposure, 5 μM ¹³C tyrosine was added 1 hour after ultraviolet (UV) radiation exposure. After 4 hours, DNA was isolated and adducts determined as an increase in ¹³C enrichment over baseline after combustion and isotope ratio mass spectrometry. Adducts of tyrosinase-derived species upon DNA cause an increase in the delta value, establishing that ultraviolet (UV) radiation exposure causes a significant increase in DNA adducts, and that tyrosinase inhibition with PTU greatly decreases DNA adducts, confirming predictions.

While the disclosure is susceptible to various modifications and alternative forms, specific exemplary embodiments thereof have been shown by way of example in the drawings and have herein been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

What is claimed is:

1. A method of preventing melanoma of the skin after ultraviolet (UV) radiation exposure comprising:
using a tyrosinase inhibitor agent to prevent the damage or death of melanocytes.

2. The method of claim 1, wherein said step of using a tyrosinase inhibitor agent includes applying said tyrosinase inhibitor agent to the skin.

3. The method of claim 1, wherein said step of using a tyrosinase inhibitor agent includes ingesting said tyrosinase inhibitor agent.

4. A system of preventing melanoma of the skin after ultraviolet (UV) radiation exposure comprising:
a tyrosinase inhibitor agent to prevent the damage or death of melanocytes.

5. The system of claim 4, wherein said tyrosinase inhibitor agent is applied to the skin.

6. The system of claim 4, wherein said tyrosinase inhibitor agent is ingested.

7. A method of preventing melanoma of the skin prior to ultraviolet (UV) radiation exposure comprising:
using a tyrosinase inhibitor agent to prevent the damage or death of melanocytes.

8. The method of claim 7, wherein said step of using a tyrosinase inhibitor agent includes applying said tyrosinase inhibitor agent to the skin.

9. The method of claim 7, wherein said step of using a tyrosinase inhibitor agent includes ingesting said tyrosinase inhibitor agent.

10. A system of preventing melanoma of the skin prior to ultraviolet (UV) radiation exposure comprising:
a tyrosinase inhibitor agent to prevent the damage or death of melanocytes.

11. The system of claim 10, wherein said tyrosinase inhibitor agent is applied to the skin.

12. The system of claim 10, wherein said tyrosinase inhibitor agent is ingested.

13. A method of preventing melanoma of the skin during ultraviolet (UV) radiation exposure comprising:
using a tyrosinase inhibitor agent to prevent the damage or death of melanocytes.

14. The method of claim 13, wherein said step of using a tyrosinase inhibitor agent includes applying said tyrosinase inhibitor agent to the skin.

15. The method of claim 13, wherein said step of using a tyrosinase inhibitor agent includes ingesting said tyrosinase inhibitor agent.

16. A system of preventing melanoma of the skin during ultraviolet (UV) radiation exposure comprising:
a tyrosinase inhibitor agent to prevent the damage or death of melanocytes.

17. The system of claim 16, wherein said tyrosinase inhibitor agent is applied to the skin.

18. The system of claim 16, wherein said tyrosinase inhibitor agent is ingested.
